(19) United States
(12) Patent Application Publication
(10) Pub. No.: US 2013/0224883 A1
(43) Pub. Date: Aug. 29, 2013

(54) Method for recognizing interferences in in vitro diagnostic assays

(71) Applicant: Juergen Patzke, Marburg (DE)

(72) Inventor: Juergen Patzke, Marburg (DE)

(73) Assignee: Siemens Healthcare Diagnostics Products GmbH, Marburg (DE)

(21) Appl. No.: 13/776,759

(22) Filed: Feb. 26, 2013

(30) Foreign Application Priority Data
Feb. 29, 2012 (EP) 12157414

(51) Int. Cl. G01N 33/68

(52) U.S. Cl. CPC G01N 33/68
USPC 436/501

(57) ABSTRACT

The present invention is in the field of in vitro diagnostics and relates to a method for recognizing interfering disruptive factors, such as heterophile antibodies or rheumatoid factors for example, in a sample.

This application claims the benefit of European Patent Application No. 12157414 filed on Feb. 29, 2012, the disclosure of which is incorporated, in its entirety, by this reference.

The present invention is in the field of in vitro diagnostics and relates to a method for recognizing interferences in a sample.

It is known that human sample material (including plasma or serum) may contain anti-immunoglobulin antibodies against animal immunoglobulins (heterophile antibodies) and/or against human immunoglobulins (rheumatoid factor, RF). Said antibodies present in the sample material can adversely affect in vitro diagnostic detection methods, in particular immunoassays, by reacting nonspecifically with one or more functional components used in the detection method (e.g., antibodies). This results in falsely excessively high assay results or, more rarely, in falsely excessively low assay results.

The first indication that a sample might contain interfering antibodies or other disruptive factors usually consists in an unexpected assay result which is possibly inconsistent with other clinical results for the patient.

To avoid interferences owing to sample-intrinsic antibodies, so-called blocking substances, which are intended to neutralize the action of the sample-intrinsic antibodies in the assay mix, are frequently used. The blocking substances used are, for example, mixtures of various polyclonal IgG immunoglobulins, polymerized IgG immunoglobulins, mixtures of monoclonal antibodies, monoclonal anti-human IgM immunoglobulins and the like (for a review, see Kricka, L. J., Human anti-animal antibody interferences in immunological assays. Clin. Chem. 1999, 45(7):942-956). The blocking substances can be contained in a reagent used for carrying out the detection method. Even though a range of commercially available blocking substances is available, it is nevertheless unclear whether the use of a particular blocking substance ensures the neutralization of all possible interfering antibodies.

One way of checking whether a sample for which an unexpectedly high assay result was measured in a first detection method contains interfering substances is to repeat the measurement with various dilutions of the sample. It is known that the disruptive effect of interfering antibodies usually decreases nonlinearly with increasing dilution. The disadvantage is that a plurality of dilutions needs to be measured and that interfering antibodies, the disruptive effect of which decreases linearly with increasing dilution in exceptional cases anyway, are not recognized.

Another way of checking whether a sample for which an unexpectedly high assay result was measured in a first sandwich immunoassay contains interfering substances, consists in using only one of the two analyte-specific antibodies used in the sandwich immunoassay, as capture antibody and detection antibody (Boscato, L. M. and Stuart, M. C., Incidence and specificity of interference in two-site immunoassays. Clin. Chem. 1986, 32(8):1491-1495). The disadvantage is that the check needs to be carried out for each of the antibodies used in the immunoassay and that specific reagents need to be prepared for this purpose.

It is an object of the present invention to provide a method for identifying a sample containing interfering disruptive factors that is less elaborate than the methods from the prior art.

The object is achieved by testing in a second assay method a sample which provides a first assay result in a first assay method in which the sample is contacted with one or more functional components in order to detect an analyte in the sample, wherein the second assay method differs from the first assay method merely in that a functional component for detecting the analyte in the sample is not contacted with the sample.

If a quantitatively significant assay result is obtained in the second assay method, this indicates the presence of a disruptive factor which interferes with the first assay method.

The omission of a functional component of an assay system normally causes the reaction between analyte and binding partner(s), required for signal generation, not to take place. However, if a reaction generating a measurable signal occurs, this signal must be regarded as nonspecific and indicates the presence of sample-intrinsic disruptive factors. Therefore, doubt must be cast on the actual assay result for the sample, and the analyte to be assayed must be tested using preferably an alternative method.

The term “interfering disruptive factors” is to be understood to mean all sample-intrinsic factors which disrupt the qualitative or quantitative detection of an analyte in a sample by interacting with one or more components of the detection system, resulting in falsely excessively high results being measured. Examples of such disruptive factors are anti-immunoglobulin antibodies against animal immunoglobulins (heterophile antibodies), in particular human anti-mouse antibodies (HAMA), and antibodies against human immunoglobulins (rheumatoid factor, RF).

A “first assay method in which the sample is contacted with one or more functional components in order to detect an analyte in the sample” is to be understood to mean any method for qualitatively or quantitatively detecting an analyte in a sample. In particular, this is to be understood to mean methods in which the sample is contacted with at least one analyte-specific binding partner and the binding reaction is detected. Examples are, in particular, immunoassays in which antigen-antibody binding is detected.

A “second assay method which differs from the first assay method merely in that a functional component for detecting the analyte in the sample is not contacted with the sample” is to be understood to mean a method which has all the functional components and assay conditions of the first assay method with the exception of a single functional component.

A “functional component for detecting the analyte” is to be understood to mean all the components of the first assay method which are indispensable for the specific detection of the analyte in the assay system, such as, for example, an analyte-binding binding partner, for example an antibody, or a secondary binding partner which binds the analyte-binding binding partner.

In a preferred embodiment of the method according to the invention, the first assay method, in which the sample is contacted with one or more functional components in order to detect an analyte in the sample, is a particle agglutination assay for determining VWF activity. In this VWF activity assay, the sample is contacted with isolated GPIbα protein, which binds to VWF in the absence of ristocetin, and with microparticles coated with a monoclonal anti-GPIbα antibody, as functional components, and the agglutination reaction is measured (WO 2009/007051 A2).

To identify interfering disruptive factors in a sample which has been tested with the above-described VWF activity assay, the sample is tested in a second assay method, wherein the second assay method differs from the first assay method merely in that isolated GPIbα protein is not contacted with the sample. This has the advantage that a functional component is dispensed with in just one reagent. All the other components, even the buffer solution containing the functional component GPIbα protein of the first assay method, remain the same.

Alternatively, the sample can be tested in a second assay method wherein the second assay method differs from the first assay method merely in that the microparticles which are contacted with the sample are not coated with anti-GPIbα antibodies.

In another preferred embodiment of the method according to the invention, the first assay method, in which the sample is contacted with one or more functional components in order to detect an analyte in the sample, is an assay for determining factor XIII.

In an exemplary factor XIII assay, the sample is contacted with thrombin, Ca²⁺ ions, a glutamine-containing peptide and glycine ethyl ester, with NADH (nicotinamide adenine dinucleotide hydride) and with components of an NADH-dependent indicator reaction, specifically with glutamate dehydrogenase (GLDH) and α-ketoglutarate, as functional components, and the change in absorption of the assay mix is measured (EP-A2-336353).

To identify interfering disruptive factors in a sample which has been tested with the above-described factor XIII assay, the sample is tested in a second assay method, wherein the second assay method differs from the first assay method merely in that thrombin or Ca²⁺ ions or glutamine-containing peptide or glycine ethyl ester or NADH or glutamate dehydrogenase (GLDH) or α-ketoglutarate is not contacted with the sample.

In another preferred embodiment of the method according to the invention, the first assay method, in which the sample is contacted with one or more functional components in order to detect an analyte in the sample, is an assay for determining prothrombin fragment F1+2.

In an exemplary F1+2 assay, the sample is contacted with a first antibody which specifically recognizes F1+2, and with a second antibody which recognizes the immunocomplex composed of F1+2 and the first antibody, and with a first component of a signal-producing system (e.g., Chemibead) that is associated with the second antibody, and with a second component of a signal-producing system (e.g., Sensibead) that is associatable with the first antibody, as functional components, and the development of chemiluminescence in the assay mix is measured (EP-A1-2168985).

To identify interfering disruptive factors in a sample which has been tested with the above-described F1+2 assay, the sample is tested in a second assay method, wherein the second assay method differs from the first assay method merely in that F1+2-specific first antibody or immunocomplex-specific second antibody or first or second component of the signal-producing system is not contacted with the sample.

### EXAMPLES

### Example 1

### Method for Identifying Interfering Disruptive Factors in a VWF Activity Assay

The INNOVANCE® VWF Ac Test (Siemens Healthcare Diagnostics Products GmbH, Marburg, Germany) is an assay for determining VWF activity in plasma samples. To determine VWF activity, the sample is first incubated with isolated GPIbα protein, which binds to VWF in the absence of ristocetin. Subsequently, latex particles coated with a monoclonal anti-GPIbα antibody are added to the assay mix. The latex particles agglutinate depending on the binding reaction of the GPIbα protein to the VWF from the sample. The isolated GPIbα protein is an essential functional component of the detection method. Without the addition of GPIbα protein, specific particle agglutination does not take place.

Two plasma samples which were known to contain human anti-mouse antibodies (HAMA sample 1 and HAMA sample 2) were tested. A plasma pool composed of plasmas from a plurality of normal donors was used as the normal sample without any interference factors (control plasma N, Siemens Healthcare Diagnostics). In addition, a 1:3 dilution of control plasma P (a diluted plasma pool composed of plasmas from a plurality of normal donors, Siemens Healthcare Diagnostics) was tested.

### Determination of VWF Activity in the Presence of GPIbα (INNOVANCE® VWF Ac Test)

To determine VWF activity in samples exhibiting VWF activities in the normal range (HAMA sample 1 and control plasma N), 15 μL of sample, 30 μL of Owren's Veronal Buffer, 70 μL of reaction buffer, 13 μL of GPIbα reagent (buffer solution containing isolated GPIbα protein, which binds to VWF in the absence of ristocetin) and 40 μL of latex reagent (buffer solution containing latex particles coated with a monoclonal anti-GPIbα antibody) were mixed, and particle agglutination was determined by turbidimetry. With this assay setting, VWF activities are determinable in the range of 15-150% of the norm.

To determine VWF activity in samples exhibiting low VWF activity (<15% VWF; see HAMA sample 2 and diluted control plasma P in table 1), 60 μL of sample, 70 μL of reaction buffer, 13 μL of GPIbα reagent and 40 μL of latex reagent were mixed. With this assay setting, VWF activities are determinable in the range of 4-20% of the norm.

### Determination of Nonspecific Reactions in the Absence of GPIbα

To determine nonspecific reactions in HAMA sample 1 and in the normal sample (control plasma N), 15 μL of sample, 30 μL of Owren's Veronal Buffer, 70 μL of reaction buffer, 13 μL of buffer solution (without any isolated GPIbα protein, which binds to VWF in the absence of ristocetin) and 40 μL of latex reagent (buffer solution containing latex particles coated with a monoclonal anti-GPIbα antibody) were mixed, and particle agglutination was determined by turbidimetry.

To determine nonspecific reactions in samples with low VWF activity (<15% VWF; see HAMA sample 2 and diluted control plasma P), 60 μL of sample, 70 μL of reaction buffer, 13 μL of buffer solution (without any isolated GPIbα protein, which binds to VWF in the absence of ristocetin) and 40 μL of latex reagent were mixed.

The results are shown in tables 1 and 2.

TABLE 1
Particle agglutination [mU/min]

| | VWF assay | VWF assay |
|---|---|---|
| Sample | with GPIb | without GPIb |
| Control plasma N | 1244 | 0.2 |
| Control plasma P | 251 | 1.9 |
| HAMA sample 1 | 1524 | 930 |
| HAMA sample 2 | 402 | 124 |

Table 1 shows the particle-agglutination rates. While the normal samples without any interference factors (control plasma N and control plasma P) show, as expected, no significant reaction in the VWF assay without GPIb protein, the HAMA samples exhibit nonspecific high particle-agglutination rates in the VWF assay with no addition of GPIb.

TABLE 2
VWF activity [% of the norm]

| | VWF assay | VWF assay | Differ- | Alternative |
|---|---|---|---|---|
| Sample | with GPIb | without GPIb | ence | VWF assay |
| Control plasma N | 93.3 | <15 | n.a. | n.a. |
| Control plasma P | 10.8 | <4 | n.a. | n.a. |
| HAMA sample 1 | 117.8 | 71.9 | 45.9 | 29.3 |
| HAMA sample 2 | 14.1 | 7.1 | 7.0 | 10.6 |

Table 2 shows the VWF activities determined on the basis of the measured particle-agglutination rates. While the normal samples without any interference factors (control plasma N and control plasma P) exhibit no measurable VWF activity in the VWF assay without GPIb protein, the HAMA samples exhibit nonspecific VWF activities in the VWF assay with no addition of GPIb. The comparison with the actual VWF activity of the HAMA samples, which was determined using an alternative VWF assay, shows that the VWF activity measured in the VWF assay with addition of GPIb represents a falsely excessively high result.

The particle-agglutination rates “without GPIb” can also be used to calculate VWF activity with the aid of the calibration curve. In the case of the HAMA samples, the correct VWF content of the samples can be determined by subtracting the value “without GPIb” from the VWF value “with GPIb” (see the column “Difference”).

1. A method for identifying interfering disruptive factors in a sample which provides a first assay result in a first assay method in which the sample is contacted with one or more functional components in order to detect an analyte in the sample, characterized in that the sample is tested in a second assay method, wherein the second assay method differs from the first assay method merely in that a functional component for detecting the analyte in the sample is not contacted with the sample.

2. The method as claimed in claim 1, wherein the sample is contacted with isolated GPIbα protein and with microparticles coated with anti-GPIbα antibodies in a first assay method for detecting the VWF activity in the sample and wherein the sample is tested in a second assay method, wherein the second assay method differs from the first assay method merely in that isolated GPIbα protein is not contacted with the sample.

3. The method as claimed in claim 1, wherein the sample is contacted with isolated GPIbα protein and with microparticles coated with anti-GPIbα antibodies in a first assay method for detecting von Willebrand factor in the sample and wherein the sample is tested in a second assay method, wherein the second assay method differs from the first assay method merely in that the microparticles which are contacted with the sample are not coated with anti-GPIbα antibodies.
